(19) United States
(12) Patent Application Publication
(10) Pub. No.: US 2002/0046755 A1
(43) Pub. Date: Apr. 25, 2002

(54) Gas-supplying device

(76) Inventor: Torsten De Voss, Hvidovre (DK)

Correspondence Address: WINSTON & STRAWN, 200 PARK AVENUE, NEW YORK, NY, 10166-4193, US

(21) Appl. No.: 10/014,188

(22) Filed: Nov. 13, 2001

Related U.S. Application Data
(63) Continuation of application No. PCT/DK00/00288, filed on May 29, 2000.

(30) Foreign Application Priority Data
May 28, 1999 (DK) PA 1999 00757

(51) Int. Cl. A62B 7/00; A61M 16/00

(52) U.S. Cl. 128/207.18; 128/204.18

(57) ABSTRACT

The present invention relates to a device for supplying a gas e.g., oxygen—to a person's respiratory tract, especially the upper nasal airways. According to a first aspect of the invention, a device for supplying gas to the airways of a person is provided, the device having at least one inlet opening for receiving a gas and at least two outlet openings adapted for supplying the gas to the person's nostrils. The device is configured such that the position of the outlet openings can be adjusted relative to each other. According to a second aspect of the invention, the device comprises at least one inlet opening for receiving a gas and at least first and second tubular members having first and second outlet openings at the distal ends thereof. The tubular members are adapted for placement in the person's nostrils for supplying gas thereto, are inclined towards each other, and adapted to elastically engage each side of the nasal septum as they are urged apart by the nasal septum in their inserted position.

### CROSS-REFERENCE TO RELATED APPLICATIONS

This application is a continuation of international application PCT/DK00/00288 filed May 29, 2000, the content of which is expressly incorporated herein by reference thereto.

### BACKGROUND ART

The present invention relates to a device for supplying a gas—e.g., oxygen, nitrogen or any other gas or mixture of gasses—to a person's respiratory tract, especially the upper nasal airways. Such a device typically comprises what is referred to as a nasal cannula having at least one inlet and two outlets wherein the two outlets are being mounted and shaped in order to fit into the nostrils of a person. The device to which the invention relates may further comprise supply hoses for feeding said inlets with a gas, as well as a manifold for merging the two supply hoses into a single supply hose for connecting with a gas source.

As stated above, the present invention relates to devices intended primarily, but not exclusively, to be placed in the nostrils of a person. At the apical entrance to the nose the nostrils are partitioned by the nasal septum to divide the nasal cavity into right and left halves. The proximate portion of the septum is bony, while distally the septum consists of cartilage which becomes progressively more flexible toward the apex. The nasal cavity is lined by a mucous membrane.

The devices to which the present invention relates may generally be used whenever a gas is to be supplied to person in a non-forced manner, i.e., the respiration relies upon the persons natural respiration, this in contrast to situations in which persons are placed in respirators by which a gas is forced into the lungs of the person. Such devices are typically used to supply, for example, additional oxygen to bedridden patients with impaired lung or vascular function but may also be used by person with sleep disorders or during certain kind of demanding sports—e.g., climbing or flying. Indeed, such devices may also advantageously be applied for e.g., taking samples of the expiratory gases of a person.

Typically, two types of devices are used for supplying gas to the nasal passageways of a person, either very simple single-tube catheters which are simply placed in one of the nostrils and fixed to hold it in place by a suitable means, for example by a soft cuff-member mounted on the distal end of the catheter and/or by taping the catheter to the person.

The second type of device, to which the present invention relates, comprises two outlets arranged in order to fit into the nostrils of a person. Typically, such a device has a main body, or nasal cannula, with two upstanding tubular members spaced and dimensioned to be placed in the nostrils of the person, together with one or two hoses which function both as the gas supply means for the main body as well as the mounting means for fixing the main body relative to the nostrils of the person.

In such a device the nasal cannula is normally being held in place by tying together the gas feeding hoses behind the neck of the person, meaning that the hoses are held in position both at the front and at the back of the person.

This method of fixing the main body results in a number of problems. As the person moves the head, there is a tendency that the main body also moves relative to the nostrils which may result in the gas outlets becoming more or less displaced. If the main body is displaced or kinked to such a degree that one of the outlets is displaced from the nostril, this may result in a severe shortage of gas supplied to the person. For a device in which the two outlets are interconnected, the gas may leave the main body where the outlet pressure is the lowest, resulting in a gas supply to the patient which is less than half the desired amount. Also, as the resistance to breathing through the two nostrils varies over the day, only the nostril with the high resistance may be supplied with gas resulting in a severe shortage in the gas supply.

Another reason for gas cannula kinking may result from anatomical reasons, as it is a common experience that it is sometimes almost impossible to properly place and affix the two outlets correctly in the nostrils, such that a few movements of the head result in a kinking and misplaced cannula.

Apart from the above problems, even a correctly placed gas cannula may result in problems over time. The nasal cavity is lined by a mucous membrane which during a prolonged time of use of the cannula may be damaged. This is due both to the mechanical impact when the cannula touches the same area of the mucous membrane during normal movements of the head, as well as to the outstreaming gas also being directed towards the same area of the mucous membrane.

A further problem associated with the prior art devices is that they normally are supplied in different sizes to fit different groups of users, i.e., babies, children, females and males.

Thus, the present invention now provides a device for supplying gas to a person which alleviates one or more of the above-identified problems, and which, preferably, does not limit the movement of the person carrying the device.

### SUMMARY OF THE INVENTION

The invention relates to a device for supplying gas to a person, in which two inlet openings are adjustable relative to each other, either manually adjustable in order to adapt to the nostrils of a person, or “automatically” adjustable to adapt to the nostrils of a person under static as well as dynamic conditions.

According to a first aspect of the invention, this device comprises at least one inlet opening for receiving a gas, and at least two outlet openings adapted for supplying the gas to the nostrils of the person. In addition, the device is configured such that the position of the outlet openings can be adjusted relative to each other.

In a preferred embodiment, the device comprises two tubular bodies which can be axially adjusted with respect to each other, this allowing the distance by which the tubular bodies protrudes into the nostrils to be adjusted.

In a further preferred embodiment, the tubular members are a pair of flexible hoses, mounted to a connector body in sliding, frictional engagement, this allowing for simple yet reliable axial adjustment between the two hoses.

In yet another preferred embodiment, the tubular members are inclined towards each other, this allowing the tubes or hoses to elastically engage the nasal septum thereby holding the device in place.

In a further preferred embodiment, the tubular members are pivotably connected to each other by a hinge member, this allowing for adjustment of the distance between the outlets as well as gripping action.

According to a second aspect of the invention, a device for supplying gas to the airways of a person is provide. This device comprises at least one inlet opening for receiving a gas, at least first and second tubular members having first and second outlet openings at the distal ends thereof, the tubular members being adapted for placement in the nostrils of the person for supplying gas thereto, wherein the tubular members are inclined towards each other, and adapted to elastically engage each side of the nasal septum as they are urged apart by the nasal septum in their inserted position. The elastic properties may either be provided by the tubular members themselves, for example in the form of flexible, elastic hoses, or by means interconnecting the two tubular members.

In a preferred embodiment, each tubular member may have protruding abutment surfaces. The distance between the abutment surfaces of the two tubular members (with or without additional abutment means) should in general be smaller than the normal thickness of a person's nasal septum. In a preferred embodiment this distance is very small, between 0 and 10 mm, more preferably between 0 and 5 mm, this allowing the device to be used with most persons. The internal diameter of the tubular members is preferably smaller than 10 mm and more preferably smaller than 5 mm.

Upon insertion into the nostrils, the two tubular members may be moved away from each other—or in general, the distance between the two abutment surfaces may be widened—and after the nasal cannula is in place in the nostrils the elastic properties of the device will attempt to recover the shape of the nasal cannula prior the insertion and thereby apply force from the abutment surfaces of tubular members to the nasal septum retaining the nasal cannula in position in the nostrils. In this way a single or eventually two sizes will fit most people.

In a preferred embodiment of the invention, the nasal cannula comprises at least one gas hose wherein the at least one outlet is formed by the at least one gas hose. By this way of inserting a gas hose directly into the nostrils, the nasal cannula according to the present invention can be manufactured in a simple and cheap way still providing the same functional benefit of being supported by the nasal septum. In another preferred embodiment of the invention the nasal cannula consist of two gas hoses held together by means of a connecting member, in an angle enabling support of the nasal cannula on the nasal septum.

In order to ensure equalization of the pressure and thereby the flow of the gas in the two outlets, the outlets can be interconnected by a tube. The internal diameter of the interconnecting tube may be balanced with or dimensioned to the foreseen pressures. A throttle means such as a valve can be mounted in the interconnecting tube in order to adjust the flow according to the supply pressure.

In a further preferred embodiment of the nasal cannula, the interconnecting tube comprises at least one opening for letting gas out to the ambience, so that gas that enters the nasal cannula through the outlets may leave the nasal cannula through said at least one opening in the interconnecting tube. This will ease the user when exhaling. Preferably, in such a situation, the supply of gas may be regulated by throttle means

In order to provide a comfortable fit, all parts of the device may be made of soft and flexible materials such as silicone rubber, nitrile rubber or any other kind of rubber or material having similar elastic properties.

In order to provide a snug fit even though the distances between the nostrils may vary from person to person, i.e., the thickness of the nasal septum, the distance between the two outlets may be adjustable. This may for instance be achieved by means of the spring effect given by a combination between the design of the nasal cannula and the selected material and/or by means of an adjustable spacer between the two outlets. The nasal cannula may furthermore be made in two pieces held together by an adjustable fixture.

As described above, the outlets may have protruding abutment surfaces, for example in the form of a bulged shape for fitting the outlets to the nostrils. The size of the bulges may be chosen such that they fit different categories of users such as adults or children. In such situations adults normally requires bulges having a larger external diameter, especially in the case where the bulge is rounded. Presently preferred is an olive-shaped bulge.

The bulges may preferably be made as separate parts to be mounted on the outlet members of the nasal cannula. In this case the material for the bulges preferably may be selected from a group of plastic/rubber materials or materials having a similar elasticity in order to provide a comfortable and “soft” pressure against the nasal septum.

In another aspect, the invention relates to a supply hose for supplying gas to the at least one inlet of the nasal cannula. In an embodiment of the nasal cannula according to the invention, in which only one inlet is provided, one supply hose may be used, whereas if two inlets are provided in the nasal cannula according to the invention, two supply hoses may be used, which may be connected each other by a manifold. The supply hose or hoses may be made of silicone rubber, nitrile rubber or any other kind of rubber or material having similar elastic properties.

In another aspect of the invention, the supply hose provides an easy and comfortable support around an ear of the user. This may be achieved by coiling a part of the supply hose e.g., in a radius between, for example, 30 mm and 40 mm. The supply hose may be coiled up to 5 times, preferably 2 to 4 times, and more preferably 0.5 times.

In another embodiment, the coiling of the tube may be performed in such a way that the shape of the coil is similar to the shape of the ear. The used material is treated or cured so as to ensure that it always recoils to the coiled shape after unwinding. This treatment may preferably be a heat treatment.

The purpose of the manifold is to merge at least two supply hoses into one supply hose. The manifold can be made of any material, with silicone rubber or similar rubber materials being preferred. The manifold may be dimensioned in relation to the pressure of the supply source and can be made with a throttle valve in order to adjust the supply pressure.

All parts of the device can be made using a variety of ordinary moulding techniques such as blow moulding, injection moulding or extrusion. Furthermore, each part of the device can be made in separate pieces and be glued, welded or pressed together.

### BRIEF DESCRIPTION OF THE DRAWING FIGURES

A detailed description of preferred embodiments of the invention now follows in conjunction with the appended figures, in which:

FIG. 1 shows a traditional prior art device for supplying a gas to the nostrils of a person;

FIG. 2 shows a first embodiment according to the present invention in which a tubular extension member is mounted in sliding engagement in a tubular member;

FIG. 3 shows a further embodiment of the present invention in which a flexible hose is mounted through an opening in a main body;

FIG. 4 shows an embodiment of the present invention of the same general type as depicted in FIG. 1 but with tubular members inclined towards each other;

FIG. 5a shows a further embodiment of the present invention in which two hose members are mounted on a common connecting member;

FIG. 5b shows the same device as in FIG. 5a but with abutment means mounted on the distal end of each of the hoses;

FIG. 6 shows a number of different configurations for the abutment means of FIG. 5b;

FIG. 7 shows an embodiment comprising two tubular members pivotably connected to each other by a hinge member;

FIG. 8 shows an embodiment of the invention in which a nasal cannula comprises two interconnected, curved tubular members;

FIGS. 9 and 10 show side and top views, respectively, of the device in FIG. 8;

FIG. 11 shows a hose connected to a cannula of the type shown in FIG. 8, the hose being coiled a number of times to provide a better fit behind an ear of the user;

FIG. 12 shows a cannula according to the invention comprising a connecting manifold;

FIG. 13 shows an embodiment of the invention is which the tubular members are connected via an interconnecting tube comprising a number of ventilating holes; and

FIG. 14 shows an embodiment in which the nasal cannula comprises two gas hoses held together by an integrally formed connecting member.

### DETAILED DESCRIPTION OF THE PREFERRED EMBODIMENTS

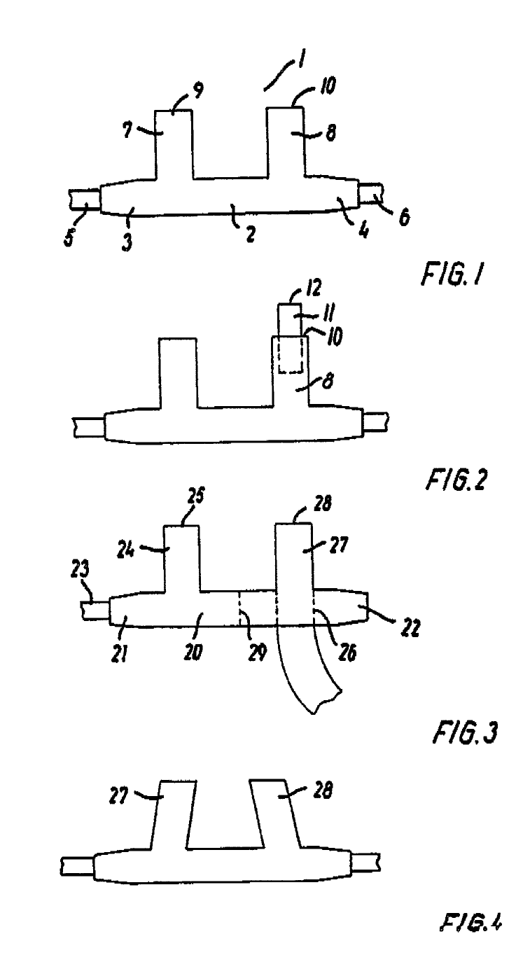

FIG. 1 shows a traditional prior art device for supplying a gas to the nostrils of a person. This device, which is also called a nasal cannula, is generally designated by 1. The cannula 1 comprises a main body in the form of a tubular body 2 having first and second open ends 3,4 to which are attached hose members 5,6 respectively, for supplying a gas to the internal of the tubular body 2. If the first and second ends 3,4 are in communication with each other, the device may be used with only a single hose, the opposite end of the main body being either free or serving as attachment for another type of member, i.e., for mounting the device on the person. Protruding from the main body and in communication with the internal thereof are mounted two tubular members 7,8 en general alignment and having distal openings 9,10 at the free ends thereof.

FIG. 2 shows a first embodiment according to the present invention in which a tubular extension member 11 with a distal opening 12 is mounted in sliding and frictional engagement in the second tubular member 8 through the opening 10. Means (not shown) is provided preventing the extension member 11 from being pulled out of the tubular member 8. In the shown embodiment only a single extension is provided, however, both of the tubular elements 7,8 could be provided with extensions if desired. The extension member 11 serves as a means for effectively varying the length of the second tubular member 8, this allowing the device to be adapted to the specific needs of a given person. For example, if the device when mounted has a tendency to kink, the extension member 11 is pulled out such that both of the distal openings 9,12 are situated at the same position in the respective nostril, which will ensure both that the device stays in the nostrils as well as gas is supplied evenly.

FIG. 3 shows a further embodiment of the present invention in which a cannula comprises a main body in the form of a tubular body 20 having a first open end 21 to which are attached a hose member 23 for supplying a gas to the internal of the tubular body 20, and a second closed end 22. Protruding from the main body 20 and in communication with the internal thereof is mounted a tubular member 24 having a distal opening 25 at the free end thereof.

Through a bore 26 in the body 20 is mounted a flexible hose 27 in sliding and frictional engagement with the body and with an opening 28 at the distal end thereof. A partition wall 29 prevents gas from leaking from the bore 26, but it would also be possible to use a body 20 with a solid right (in the figure) portion. The adjustable hose is used in the same way as explained above with reference to FIG. 2.

FIG. 4 shows an embodiment of the present invention of the same general type as described with reference to FIG. 1 with the sole difference that the first and second tubular members 27, 28 are inclined towards each other, this allowing for the nasal cannula to elastically engage and “grip” the nasal septum of a user. This aspect of the present invention will be explained in greater detail below.

FIG. 5a shows a further embodiment of the present invention in which two hose members are mounted on a common connecting member. More specifically, the device comprises a connecting member 30 having bores 31,32 at opposite ends thereof. Through the bores 31,32 in the body 30 flexible hoses 33,34 are mounted in sliding and frictional engagement with the body and with openings 35,36 at the distal ends thereof, thereby forming nostril engaging tubular members. As can be seen the connecting member is bend corresponding to the middle portion 37 thus resulting in the hoses being arranged inclined towards each other. Indeed, the inclined arrangement of the hoses could also be provided by bores 31,32 having axes inclined towards each other. The shown embodiment assures that both of the hoses 33,34 can be easily adjusted lengthwise in order to provide an adjustment means for the position of the openings 35,36 with respect to each other as well as to the connecting member. Instead of a frictional engagement other locking means could be used in order to fixate the hoses in the desired position.

Further, the resilient nature of the flexible hoses 33, 34 in combination with the inclined orientation assures that the distal ends of the hoses can elastically engage and “grip” the nasal septum of a user. As a standard hose can be used in combination with the very simple connecting bridge element, the device can be manufactured very cost-effective. As described with reference to FIG. 14 below, the connecting member may also be integrally formed with the hoses. As appears from the figures, a device of this general configuration can be made relatively small, this in contrast to the rather bulky prior art devices.

A number of modifications would be possible for the embodiment of FIG. 5a; for example, the connecting member may be formed integrally with or bonded to the hose members which would result in a cannula which cannot be taken apart or misadjusted, but would dispense with the possibility of axial and lengthwise adjustment.

In order to provide a comfortable as well as secure “grip” on the nasal septum, the portion of the distal ends of the tubular members, or the hose members, facing towards each other may be provided with protruding abutment means adapted for engaging the nasal septum, however, for most practical purposes, the abutment means are formed by rotation-symmetrical elements arranged circumferentially on the tubular members in the vicinity of their respective outlet openings. In FIG. 5a an embodiment corresponding to the embodiment of FIG. 5b is shown, however, with additional abutment means 38 mounted on the distal end of each of the hoses 33,34.

When assembling a given device of the type shown in FIG. 5b, it is clear that by varying the dimensions and the materials of the different members, i.e., the hoses, the connecting member and the bulges, many different variant can be achieved in a very simple and cost-effective way.

It would also be possible to supply kits in which, for example, different connecting members and different bulges are contained, this allowing the user or a nurse or doctor to “custom-design” a nasal cannula to suit a given person.

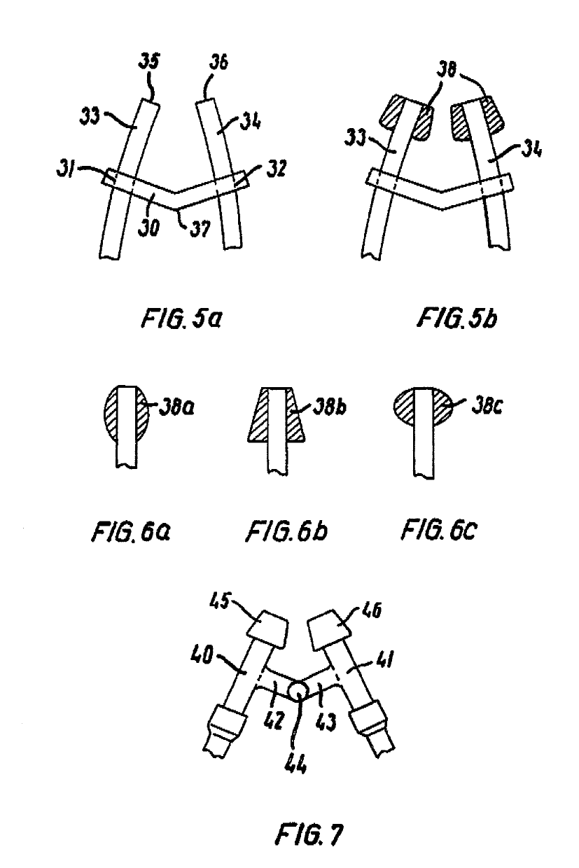

In FIGS. 6a-c a number of different configurations for such abutment means, or bulges, are shown; olive-shaped bulge (38a) as in FIG. 6a, conical bulge (38b) as in FIG. 6b and spherical bulge (38c) as in FIG. 6c. Preferably the diameter of the bulge for use with a cannula of the present invention is less than 20 mm, more preferably less than 15 mm and most preferably less than 10 mm. The bulge members can either be detachably or fixedly mounted on the tubular members.

FIG. 7 shows an embodiment comprising first and second tubular members 40, 41 each having side members 42, 43 pivotably connected to each other by a hinge member 44.

At the distal open ends of the tubular members abutment means 45,46 are mounted and at the proximal ends of thereof flexible hose members are attached. In this embodiment the engaging action is provided by the hinge member allowing the tubular members, which then may be manufactured of a relatively rigid material, to pivot against each other when a light outwards pulling force is applied by the hose members, for example when they are connected to each other behind the neck of the user. If desired, the hinge member may also be provided with a locking means allowing the user to a pre-set and lock an angle between the two tubular members.

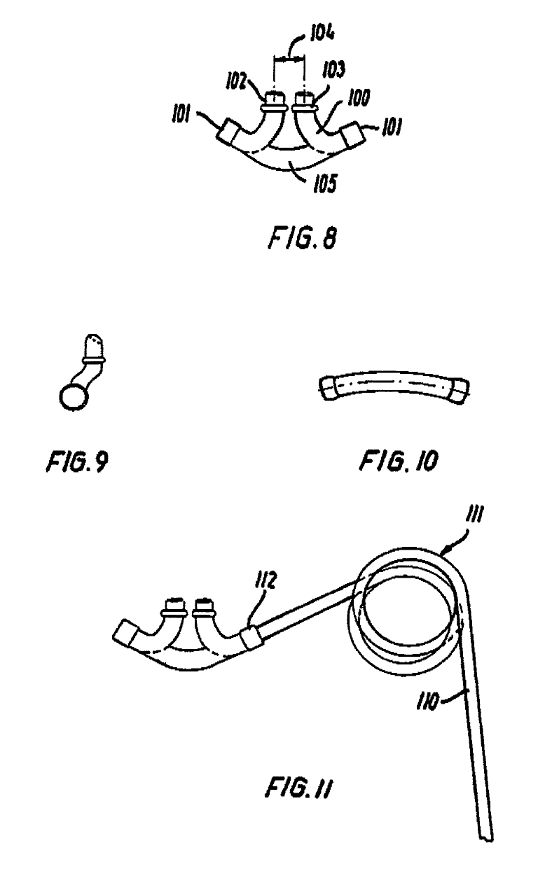

A further preferred embodiment is shown in FIGS. 8-10 in which FIG. 8 shows a nasal cannula with two interconnected, curved tubular members 100 providing two inlets 101 and two outlets 102. The nasal cannula has a shape as shown, where the distance 104 between the two outlets matches the thickness of the nasal septum of an average person. The outlets 102 form a tubular member for mounting of bulges 103. The two inlets 101 are placed substantially perpendicular to the outlets 102 either in one or, as shown, both sides. A connecting member 105 allowing gas to flow between the outlets 102 interconnects the two tubular members. In an alternative embodiment, the connecting member 105 may be solid, thus preventing gas flow between the two tubular members.

The nasal cannula is made of a flexible material allowing it to adjust to the nasal properties of a user. Engagement bulges 103 are preferably mounted on the outlets 102. The bulges can be made and supplied in different sizes in order to fit different persons. FIGS. 9 and 10 show side and top views, respectively, of the device in FIG. 8

Referring to FIG. 11 a hose 110 having one end fitting to the nasal cannula and one end fitting to a manifold 113 is coiled a number of times at a section 111 roughly in the middle of its entire length, so as to provide a better fit behind an ear of the user. The connection 112 between the nasal cannula and the hose is made by pressing the hose into the tube so that the inner surface of the inlet presses against the outer surface of the hose and only by the pressure between these two surfaces the connection is sealed. Alternatively the connection can be made by gluing the two pieces together. It would also be possible to mount a hose member on the outside of the inlet member. As seen in FIG. 12, at the other end the hose 110 is connected to a manifold 130 having one or more outlets 114 and one inlet 115 further connected to a hose 116 for supplying a gas, which typically comprises a connector (not shown) for connecting the hose to a gas outlet. The connection between the different hoses and the manifold are made by pressing the hoses into the outlets of the manifold so that the outer surface of a hose is pressed against the inner surface of the corresponding outlet of the manifold. Alternatively the connection can be made, for example, by gluing or welding the two pieces together.

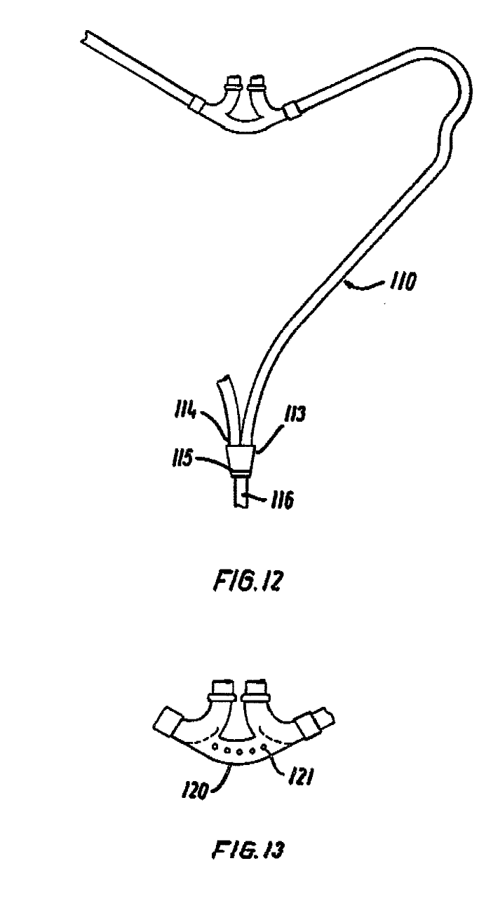

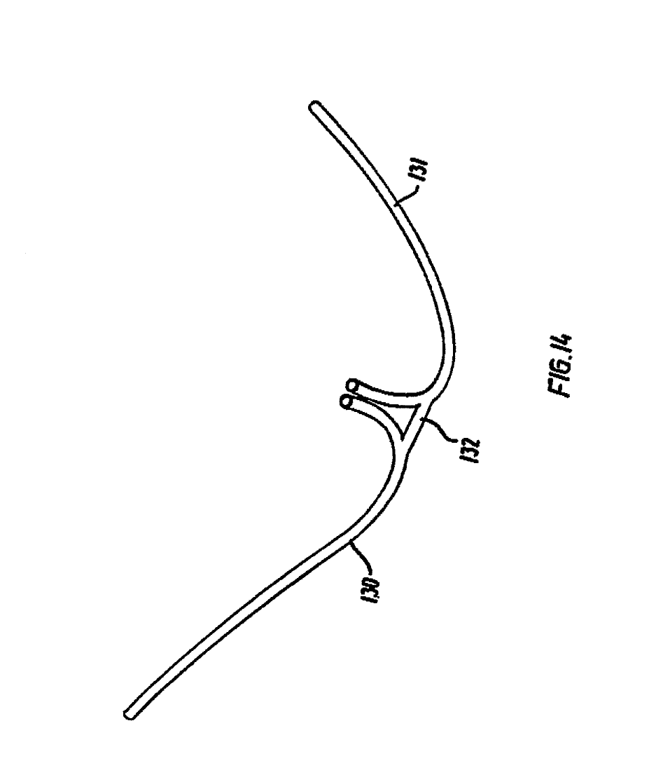

Referring to FIG. 13, in another preferred embodiment of the invention the two tubular members are connected with an interconnecting tube 120 allowing gas to flow between the two tubular members. A preferred embodiment is shown in FIG. 13 in which the interconnecting tube comprises a number of holes 121 allowing gas to leave the nasal cannula through the interconnecting tube 120. The intention with these holes is to allow the exhaled air from a patient to enter through the outlets and leave the nasal cannula through the interconnecting tube thus allowing the patient to exhale with less resistance.

A further preferred embodiment of the invention is shown in FIG. 14 in which the nasal cannula comprises two gas hoses 130, 131 being held together by an integrally formed connecting member 132, or fixture, in a way enabling the end of the gas hoses to form the outlets of the nasal cannula. The connecting member may be attached to the hoses by any suitable means, such as gluing, or by moulding of the member onto the hose members. This embodiment constitutes thus fewer parts and provides for cost-effective assembling. Such a fixture holding the two gas hoses in a preset angle ensures that the outlets, the end of the two hoses, can be elastically supported on the nasal septum. An abutment member for fitting the outlets to the nostrils are desirable, and can be mounted on the distal end of the gas hoses as separate parts. As seen in FIG. 15 the gas hoses can have a pre-set coiled shape to provide for a comfortable fit behind the ears of the user.

The use of a nasal cannula according to the present inventions and as shown with reference to FIGS. 5 to 15 will now be shortly described. The distal ends with the outlet openings 35, 36 (see FIG. 5a) of the tubular members 33, 34 are placed in the nostrils of the user after the ends have been forced away from each other, i.e., the distance 104 (see FIG. 8) is increased. This is performed by hand. After the distal ends have been placed into the nostrils, the forces exerted by the tubular members, either provided by the elastic properties of the tubular members (or hoses) or by the pulling action as in the FIG. 7 embodiment. It is evident that elastic properties should be chosen which ensure that the mucous membranes are not harmfully affected yet ensure a proper fixing action of the device on the nasal septum. It is also clear that this to a certain degree can be assured by properly choosing form, size and material for the optional bulge elements. For the embodiments in which one or both of the tubular members are lengthwise adjustable, a further protection of the mucous membranes is provided as the contacting points of the device can be re-adjusted at intervals, this protecting both against mechanical damage as well as the drying-out action of the out-streaming gas.

What is claimed is:

1. A device for supplying gas to the airways of a person, the device comprising,
at least one inlet opening for receiving a gas,
first and second flexible tubular members terminating at distal ends and having first and second outlet openings, respectively, in the distal ends thereof, the openings being adapted for supplying the gas to the nostrils of the person,
a body for connecting the tubular members, and
bores formed at opposite ends of the connection body for mounting the flexible hoses in a sliding and frictional engagement.

2. The device of claim 1, wherein the first and second tubular members define first and second axes along their lengths, and wherein the tubular members can be axially adjusted with respect to each other.

3. The device of claim 2, wherein the first and second tubular members are inclined along their axes towards each other.

4. The device of claim 1, wherein the flexible hoses are axially adjustable relative to the connector body.

5. The device of claim 4, wherein the first and second tubular members are flexible and adapted to elastically engage each side of the person's nasal septum as they are urged apart by the nasal septum in their inserted position, and wherein the forces exerted by tubular members when elastically engaging the nasal septum allow for sustained placement in the person's nostrils without substantially harming his or her nasal mucous membrane.

6. The device of claim 1, wherein the first and second tubular members are able to pivot towards each other.

7. The device of claim 1, the distal ends of the tubular members prior to insertion thereof in the person's nostrils are separated by a distance of less than 20 mm.

8. The device of claim 1, further comprising spacer means for enabling adjustment of the distance between the two outlet openings.

9. The device of claim 1, further comprising throttling means for controlling the flow of gas through the device.

10. The device of claim 1, further comprising hose means adapted for connecting the device to a gas supply outlet.

11. The device of claim 1, further comprising a supply hose for supplying a gas to the at least one inlet opening, the hose being a separate component or an integral part of the device, wherein a portion of the hose is coiled, and wherein the hose is made of a material that has been treated so as to ensure that it substantially recovers its coiled shape after unwinding.

12. The device of claim 1, further comprising two supply hoses interconnected by a manifold, the device comprising means for adjusting the flow of gas through the manifold.

13. The device of claim 1, wherein the tubular members at the portions of their distal ends facing towards each other comprise protruding abutment means adapted for engaging the nasal septum.

14. The device of claim 13, wherein the abutment means are formed by elements arranged circumferentially on the tubular members in the vicinity of their respective outlet openings.

15. A device for supplying gas to the airways of a person, the device comprising;
at least one inlet opening for receiving a gas,
first and second flexible tubular members terminating at distal ends and having first and second outlet openings, respectively, in their distal ends, the openings being adapted for supplying a gas to a nostrils of the person, and
a connecting body for interconnecting the first and second tubular members thus, allowing gas to flow between them,
wherein the connecting body comprises at least one opening for out-letting gas so that gas that enters the nasal cannula through the openings may leave the nasal canula through the at least one opening in the connecting body.

16. The device of claim 15, wherein the first and second tubular members are inclined along their axes towards each other.

17. A device of claim 15, wherein the first and second tubular members are flexible and adapted to elastically engage each side of the person's nasal septum as they are urged apart by the nasal septum in their inserted position, and wherein the forces exerted by tubular members when elastically engaging the nasal septum allows for sustained placement in the persons' nostrils without substantially harming his or her nasal mucous membrane.

18. The device of claim 15, the distal ends of the tubular members prior to insertion thereof in the person's nostrils are separated by a distance of less than 20 mm.

19. The device of claim 15, further comprising spacer means for enabling adjustment of the distance between the two outlet openings.

20. The device of claim 15, further comprising throttling means for controlling the flow of gas through the device.

21. The device of claim 15, further comprising hose means adapted for connecting the device to a gas supply outlet.

22. The device of claim 15, further comprising a supply hose for supplying a gas to the at least one inlet opening, the hose being an separate component or an integral part of the device, wherein a portion of the hose is coiled, and wherein the hose is made of a material that has been treated so as to ensure that it substantially recovers its coiled shape after unwinding.

23. The device of claim 15, further comprising two supply hoses interconnected by a manifold, the device comprising means for adjusting the flow of gas through the manifold.

24. The device of claim 15, wherein the tubular members at the portions of their distal ends facing towards each other comprise protruding abutment means adapted for engaging the nasal septum.

25. The device of claim 24, wherein the abutment means are formed by elements arranged circumferentially on the tubular members in the vicinity of their respective outlet openings.
